(19) United States
(12) Patent Application Publication
(10) Pub. No.: US 2015/0118689 A1
(43) Pub. Date: Apr. 30, 2015

(54) SYSTEMS AND METHODS FOR WHOLE BLOOD ASSAYS

(71) Applicant: Quidel Corporation, San Diego, CA (US)

(72) Inventors: Richard L. Egan, Oceanside, CA (US); Larry Thomas Mimms, Poway, CA (US); Ferda Yantiri-Wernimont, Oceanside, CA (US)

(21) Appl. No.: 14/522,314

(22) Filed: Oct. 23, 2014

Related U.S. Application Data
(60) Provisional application No. 61/895,330, filed on Oct. 24, 2013.

(51) Int. Cl. A61B 5/145; A61B 5/00; G01N 33/569; A61B 5/1455

(52) U.S. Cl. CPC A61B 5/14535; A61B 5/1455; A61B 5/7278; A61B 5/7203; G01N 33/56966; G01N 2333/805
USPC 435/7.1; 600/309; 600/328

(57) ABSTRACT

A system comprised of a device for measuring hemoglobin content and/or determining a hematocrit value and/or measuring a hematocrit value, and a device for measuring or detecting an analyte, and a method for measuring or determining the presence of at least one analyte are described.

### CROSS-REFERENCE TO RELATED APPLICATIONS

This application claims the benefit of U.S. Provisional Application No. 61/895,330, filed Oct. 24, 2013, incorporated by reference herein.

### TECHNICAL FIELD

The subject matter described herein relates to systems, apparatuses, and methods for analysis of a sample to aid in medical diagnosis or detection of the presence or absence of one or more analytes in the sample.

### BACKGROUND

Assay devices for detection of an analyte in a sample are long known in the art and include detection gels, microfluidic devices, immunoassays, and the like. In particular, lateral flow immunoassay devices are routinely used for detecting the presence of an analyte in a sample. Lateral flow immunoassay devices utilized a labeled specific binding reagent that is releasably immobilized on a test strip of porous material. A liquid sample, such as a biological sample from a human or an environmental sample, is applied to one end of the porous strip and the capillary properties of the strip transports the liquid sample along the strip, releasing the labeled specific binding reagent, which binds specifically to the analyte of interest at a first binding site thereof, if present, in the sample. The labeled binding reagent is then typically captured at a test zone by a second reagent having specific binding for a second binding site of the analyte of interest. Excess labeled binding reagent is captured at a control zone, downstream of the test zone by a control reagent which binds specifically to the labeled reagent.

Such lateral flow assay devices are commercially available, for example, to detect pregnancy by the presence of a human chorionic gonadotropin (hCG) in a urine sample applied to the test device. In such tests, two signals visible by the naked eye of the user are generated. One signal is a “control” signal, and is formed by the localization of derivatized blue latex beads, The latex beads are coated with an immunoglobulin molecule and are captured by a capture antibody, deposited in a line on the test strip generally perpendicular to the direction of sample flow, the capture antibody having specific binding activity for the immunoglobulin carried on the beads. The generation of this signal informs the user that (i) neither the immunoglobulin on the latex bead, nor the capture antibody on the test stick, have been sufficiently denatured or otherwise degraded during manufacture or storage of the test kit significantly to interfere with the specific binding between the two molecules; and (ii) sufficient liquid sample has been applied to mobilize the releasably immobilized latex beads and to transport them along the test stick at least as far as the “control” zone, in which the capture antibody is located. A urine sample containing hCG contacted with the test stick in a correctly-performed assay, will cause the deposition of latex beads in both the control zone and in the test zone, resulting in the formation of two blue lines visible to the user, one line in the control zone and one line in the test zone.

For many analytes, the physiological sample used in an analyte detection assay is blood or blood derived products such as plasma or serum. Point-of-care (POC) testing is becoming more prevalent allowing testing in the home, a doctor's office, or in remote locations without the need for a laboratory. POC testing is rapid and typically less costly than testing in a laboratory setting. A significant barrier to using whole blood (e.g. using a finger stick) is the variable volume of plasma within whole blood between different individuals and/or different points of collection between the same individual. Variations in hematocrit values for whole blood samples that are used in diagnostic tests can interfere with the accurate measurement of an analyte. The volume percentage of a whole blood sample attributed to red blood cells (RBCs), i.e., hematocrit or packed cell volume, can differ by 30% or more between individuals, which results in the concentration of the analyte in a whole blood sample differing significantly due to difference in the hematocrit. Variations in the hematocrit may affect an assay by interfering with the optical signal used for detection and/or by interfering with the chemical reaction(s) used in the assay and/or obstructing the diffusion of the analyte in the sample, for example.

Previous methods to remove, reduce or account for hematocrit variability include collection of a large volume of blood (e.g. on the order of 5-10 mL or more) and centrifuging the blood to separate the blood cells from serum or plasma, which is then used for testing. Use of serum or plasma eliminates the “whole blood effect” as the RBCs are removed. Others have used elaborate systems that separate RBCs by clotting, agglutination, or filtration. For example, U.S. Pat. No. 5,306,623 describes the use of a filter on a reagent strip to separate RBCs prior to detection of glucose in a sample. However, many samples require dilution prior to testing. The dilution step requires analytical precision and traditionally the dilution is made using serum or plasma where the RBCs are removed from the sample prior to dilution.

Another method to account for hematocrit variability in an assay for determining the presence and/or amount of an analyte uses a nonlytic hypertonic salt composition to adjust the hematocrit by reducing the size of RBCs (U.S. Pat. No. 7,323,315). Others have proposed determining the concentration of an analyte in a sample using an electrochemical cell and then using a hematocrit correction factor to derive a hematocrit corrected analyte concentration for the sample (U.S. Pat. No. 6,475,372). The hematocrit correction factor is determined using a formula that is a function of the preliminary analyte concentration and a variable (γ) that is derived from variances in measurements of the electrochemical cell.

As hematocrit values vary among individuals, an accurate analyte concentration from whole blood cannot be obtained using a uniform correction factor. Accurate hematocrit corrections should ideally be performed with specifically measured hematocrit values. U.S. Patent Publication No. 2013/0052655 describes measuring a hematocrit value using the same blood sample used for measuring the analyte concentration. The hematocrit value is typically obtained using an automated hemocytometer. Hematocrit may also be measured manually using a microhematocrit where a whole blood sample is centrifuged in a suitable container, usually a tube, to separate the blood components. From this stacked cell column, the volume of RBCs can be compared to the total volume. U.S. Pat. No. 5,277,181 describes devices that optically measure hemoglobin content, which can be used to determine the hematocrit value. U.S. Pat. No. 8,130,105 describes a multi-parameter patient monitor that measures hemoglobin as one of several physiological parameters.

There remains a need for methods and systems that minimize the effect of hematocrit in analyte detection that is suitable for point of care (POC) applications. The present methods and systems provide solutions to this problem.

### BRIEF SUMMARY

The following aspects and embodiments thereof described and illustrated below are meant to be exemplary and illustrative, not limiting in scope.

In one aspect, a method for measuring or detecting an analyte in a whole blood sample is contemplated. In one embodiment, the method comprises measuring hemoglobin content of a subject; calculating a hematocrit value from the measured hemoglobin content; measuring an analyte concentration in the whole blood sample with a measurement device; and adjusting the measured analyte concentration based on the hematocrit value. In one embodiment, the hemoglobin content of the whole blood sample is non-invasively measured. In other embodiments, the hematocrit value is calculated manually or by a hemoglobin measuring apparatus or the analyte measurement device.

In a further embodiment, a method for measuring or detecting an analyte in a whole blood sample, comprises measuring a hematocrit value of the whole blood sample; measuring an analyte concentration in the whole blood sample with a measurement device; and adjusting the measured analyte concentration based on the hematocrit value. In one embodiment, the hematocrit value is measured using a non-invasive approach and in another embodiment the hematocrit value is measured using an invasive approach involving taking a blood sample.

In embodiments, the methods further include diluting the blood sample prior to measuring the hemoglobin content.

In other embodiments, non-invasively measuring the hemoglobin content comprises non-invasively measuring using a hemoglobin meter. In further embodiments, the hemoglobin meter is a pulse co-oximetry meter. In additional embodiments, non-invasively measuring the hematocrit value comprises non-invasively measuring using a hematocrit meter.

In embodiments, measuring the analyte concentration uses a device for measuring the analyte concentration, and the method further comprises entering the hemoglobin content and/or hematocrit value into the device, whereby the device automatically adjusts the analyte concentration based on the entered hematocrit value. The hemoglobin content and/or the hematocrit value may be manually entered into the device. In other embodiments, the hemoglobin or hematocrit meter may connect with the device to communicate one of the hemoglobin content and/or the hematocrit value. This connection may be a wired or wireless connection. In an embodiment, the hemoglobin content and/or hematocrit value is transmitted to the device through a wireless transmitter.

In embodiments, the analyte is at least one of vitamin D, one or more vitamin D metabolites, or one or more vitamin D derivatives or analogs. It will be appreciated that the device may measure the analyte concentration of one or more of vitamin D, vitamin D metabolites, and/or vitamin D derivatives.

In another aspect, a system for measuring or detecting an analyte in a whole blood sample, comprises a hemoglobin meter for measuring a hemoglobin content of the whole blood sample, the meter comprising a wireless transmitter; and a device for measuring or detecting the analyte. The hemoglobin meter communicates with the device to send data corresponding to the hemoglobin content to the device; and wherein the device corrects the measurement or detection of the analyte based on the transmitted hemoglobin content. In embodiments, the hemoglobin meter communicates with the device wiredly or wirelessly. In one embodiment, the hemoglobin meter communicates with the device through a wireless transmitter. It will further be appreciated that a hematocrit value may be transmitted additionally or in place of the hemoglobin content. The hematocrit value may be calculated by the hemoglobin meter and/or the device for measuring or detecting the analyte. In one embodiment, the hemoglobin meter non-invasively measures hemoglobin content of blood or hematocrit value of blood.

In a further aspect, a system for measuring or detecting an analyte in a whole blood sample, comprises a hemoglobin meter for measuring hemoglobin content of the whole blood sample, and a device for measuring or detecting the analyte. The device includes an input or interface for entering data corresponding to the hemoglobin content. The device corrects the measurement or detection of the analyte based on the entered hemoglobin content. It will further be appreciated that a hematocrit value may be entered additionally or in place of the hemoglobin content. The hematocrit value may be calculated manually or by the hemoglobin meter and/or the device for measuring or detecting the analyte. In one embodiment, the hemoglobin meter non-invasively measures hemoglobin content of blood or hematocrit value of blood.

In a further aspect, a system for measuring or detecting an analyte in a whole blood sample, comprises a hematocrit meter for measuring a hematocrit value of the sample, the meter comprising a wireless transmitter; and a device for measuring or detecting the analyte. The hematocrit meter communicates with the device through the wireless transmitter to send data corresponding to the hematocrit value to the device. The device corrects the measurement or detection of the analyte based on the transmitted hematocrit value. In other embodiments, the hematocrit meter communicates with the device wiredly or wirelessly. In one embodiment, the hematocrit meter communicates with the device through a wireless transmitter. In one embodiment, the hematocrit meter non-invasively measures the hematocrit value of blood.

In embodiments, the device includes an immunochromatographic test strip comprising a detection zone which contains an immobilized reagent capable of binding the analyte for measuring or detecting the analyte.

In other embodiments, the device comprises at least one algorithm for calculating the concentration of the analyte based on the measured or detected analyte and the data corresponding to the hemoglobin content and/or hematocrit value.

In further embodiments, the hemoglobin meter is capable of converting a hemoglobin measurement to a hematocrit value, and the transmitter transmits the hematocrit value to the device.

In embodiments, the device is capable of correcting a measurement or a detection of the analyte based on the hematocrit value.

In another aspect, a method for measuring an analyte concentration in a whole blood sample is provided. The method comprises measuring a hemoglobin content of a subject's blood or of a whole blood sample from a subject; calculating a hematocrit value from the measured hemoglobin content; measuring an analyte concentration in the whole blood sample with a measurement device; and adjusting the measured analyte concentration based on the hematocrit value.

In one embodiment measuring a hemoglobin content comprises non-invasively measuring a hemoglobin content. For example, non-invasively measuring the hemoglobin content can comprise non-invasively measuring using a hemoglobin meter. In one embodiment, the hemoglobin meter comprises software comprising an algorithm to calculate a hematocrit value from the measured hemoglobin content.

In one embodiment, measuring the analyte concentration comprises a measurement device comprising an analyte test assay and an instrument to read a signal emanating from the analyte test assay to measure concentration of analyte, and the method further comprises providing the hemoglobin content to the instrument, whereby software on the instrument adjusts the measured analyte concentration based on the hemoglobin content.

In one embodiment, the hemoglobin content is wirelessly transmitted from the meter to the measurement device.

In another embodiment, measuring the analyte concentration comprises a measurement device comprising an analyte test assay and an instrument to read a signal emanating from the analyte test assay to measure concentration of analyte, and the method further comprises providing hematocrit value to the instrument, whereby software on the instrument adjusts the measured analyte concentration based on the hematocrit value.

In one embodiment, hematocrit value is wirelessly transmitted from the meter to the measurement device.

In another aspect, a method for measuring an analyte concentration in a whole blood sample is provided. The method comprises measuring a hematocrit value of a subject's blood or of a whole blood sample; measuring an analyte concentration in the whole blood sample with a measurement device; and adjusting the measured analyte concentration based on the hematocrit value.

In one embodiment, measuring a hematocrit value comprises non-invasively measuring a hematocrit value. For example, a hematocrit value may comprise, in one embodiment, measuring a hemoglobin level and calculating a hematocrit value.

In another embodiment, non-invasively measuring the hematocrit value comprises non-invasively measuring using a hematocrit meter.

In yet another embodiment, measuring the analyte concentration comprises a measurement device comprising an analyte test assay and an instrument to read a signal emanating from the analyte test assay to measure concentration of analyte, and the method further comprises providing the hematocrit value to the instrument, whereby software on the instrument adjusts the measured analyte concentration based on the hematocrit value.

The hematocrit value, in one embodiment, is wirelessly transmitted from the meter to the measurement device.

In another aspect, a system for measuring an analyte concentration in a whole blood sample is provided. The system comprises (1) a meter selected from (i) a hemoglobin meter for measuring a hemoglobin content of a subject's blood or of the whole blood sample; and (ii) a hematocrit meter for measuring a hematocrit value of a subject's blood or of the whole blood sample; and (2) a device for measuring the analyte concentration in the whole blood sample, the device comprising an instrument with (i) a user interface to input the measured hemoglobin content or hematocrit value and (ii) software to adjust a measured analyte concentration based on the measured hemoglobin content or hematocrit value. The device reports to a user an analyte concentration adjusted by the measured hemoglobin content or hematocrit value.

In one embodiment, the device comprises an immunochromatographic test strip comprising a detection zone which contains an immobilized reagent capable of binding the analyte for detecting the analyte.

In another embodiment, the meter is a hemoglobin meter and the data corresponds to a measured hemoglobin content, and wherein the software comprises an algorithm to calculate a hematocrit value from the measured hemoglobin content.

In still another embodiment, the meter is a hemoglobin meter that comprises an algorithm to calculate a hematocrit value from the measured hemoglobin content, wherein the hemoglobin meter reports or transmits the calculated hematocrit value to the device for measuring the analyte concentration in the whole blood sample.

In yet another aspect, a system for measuring an analyte concentration in a whole blood sample is provided. The system comprises (1) a meter selected from (i) a hemoglobin meter for measuring a hemoglobin content of a subject's blood or of the whole blood sample; and (ii) a hematocrit meter for measuring a hematocrit value of a subject's blood or of the whole blood sample, the meter comprising a wireless transmitter; and (2) a device for measuring the analyte concentration in the whole blood sample, the device comprising (i) a wireless receiver to receive data corresponding to a measured hemoglobin content or a measured hematocrit value from the meter and (ii) software to adjust a measured analyte concentration based on the data. The device reports an analyte concentration adjusted by the transmitted data.

In one embodiment, the meter is a hemoglobin meter and the data corresponds to a measured hemoglobin content, and wherein the software comprises an algorithm to calculate a hematocrit value from the measured hemoglobin content.

In still another embodiment, the meter is a hemoglobin meter that comprises an algorithm to calculate a hematocrit value from the measured hemoglobin content, wherein the hemoglobin meter reports or transmits the calculated hematocrit value to the device for measuring the analyte concentration in the whole blood sample.

Additional embodiments of the present systems, apparatus and methods will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the system, apparatus or method. Additional aspects and advantages of the present systems and apparatus are set forth in the following description and claims, particularly when considered in conjunction with the accompanying examples and drawings.

### BRIEF DESCRIPTION OF THE DRAWINGS

FIG. 1 is an illustration of the sequence of events in one embodiment of an assay;

FIG. 2 is a perspective view of one embodiment of a test device, exemplified by a lateral flow immunoassay;

FIGS. 3A-3B are illustrations of a test strips enclosed in a housing sized for insertion into a drawer of an apparatus;

FIG. 4 is a top view of an exemplary test strip and the arrangement of its structural and immunochemical features for interaction with the apparatus;

FIG. 5 is front perspective view of an exemplary assay apparatus; and

FIG. 6 is a top perspective view of an exemplary apparatus showing the drawer in an open position with a lateral flow immunoassay test device inserted into the drawer.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

As used in this specification, the singular forms “a,” “an,” and “the” include plural referents unless the context clearly dictates otherwise. Thus, for example, reference to an “antibody” includes a single antibody as well as two or more of the same or different antibodies, reference to an “excipient” includes a single excipient as well as two or more of the same or different excipients, and the like.

“Hematocrit” (Ht) as used herein refers to the ratio of red blood cells (RBCs) to total blood volume. Hematocrit is often expressed as a volume percentage (%) of red blood cells (RBCs) in a blood sample.

“Hemoglobin” (Hb) is the iron-containing oxygen-transport metalloprotein in the red blood cells of vertebrates. Hemoglobin level or hemoglobin concentration refers to the amount or mass per volume of hemoglobin in the blood, and is typically measured in grams per liter (g/L) or grams per deciliter (g/dL).

### II. Assay Method

In one aspect assay methods for detecting and/or measuring an analyte in a whole blood sample are provided. Specifically, the present methods allow for detection and/or measurement of an analyte in a whole blood sample while accounting for the whole blood effect due to the presence of red blood cells (RBCs). The present methods further, or in addition, allow for dilution of whole blood samples prior to analysis.

In one embodiment, the present method takes advantage of non-invasive technology to determine the hemoglobin content or hematocrit value of a subject's blood without the need for taking a blood sample and/or using complicated and expensive laboratory techniques and equipment. By non-invasive it is intended that no instrument is required to pierce the skin of the patient to obtain a blood sample. In other embodiments, hemoglobin content is determined from a blood sample that is invasively obtained. The present methods take advantage of a good relationship between hemoglobin content in the blood and the hematocrit value. Therefore measurement of the hemoglobin content can be used to determine the hematocrit value, which can be used in turn to adjust a measured analyte concentration based on the hematocrit value. The hematocrit value is approximately three times the hemoglobin content.

The hemoglobin content may be measured using any suitable hemoglobin monitor or meter as known in the art. A review of some suitable hemoglobin meters is described in Ruckman, Jared. S., Master's Theses. Paper 75., digitalcommons.uconn.edu/gs_theses/75. In some embodiments, the hemoglobin meter uses optical or spectroscopic methods to measure a hemoglobin content.

Hemoglobin binds loosely with oxygen for transport and is referred to as oxyhemoglobin. Deoxygenated hemoglobin is the form of hemoglobin without bound oxygen. The absorption wavelength for oxyhemoglobin is about 660 nm and the absorption wavelength for deoxygenated hemoglobin is about 940 nm. Total hemoglobin generally refers at least to the combined oxyhemoglobin and deoxygenated hemoglobin. Hemoglobin also may bind carbon monoxide to form carboxyhemoglobin (CO-Hb), which prevents hemoglobin from binding to oxygen. Methemoglobin (MetHb) is a dysfunctional form of hemoglobin where the iron in the heme group is in the Fe³⁺ ferric state rather than the Fe²⁺ ferrous state of hemoglobin. In other embodiments, total hemoglobin may further include carboxyhemoglobin and/or methemoglobin measurements. In an embodiment, the meter used in the method to determine hemoglobin content of blood in a subject is a pulse oximeter, which can distinguish between oxyhemoglobin and deoxyhemoglobin using LEDs emitting at approximately 660 nm and at approximately 940 nm. Emitted light is passed through a portion of the body, usually a finger, and measured by a photodetector positioned opposite the LED. Pulse oximeters generally do not measure dyshemoglobins such as carboxyhemoglobin or methemoglobin. Accordingly, the method herein contemplates measuring total hemoglobin, oxyhemoglobin, reduced oxyhemoglobin, carboxyhemoglobin and/or methemoglobin.

In another embodiment, the meter is a pulse CO-oximeter, which uses multiple wavelengths in order to measure the different types of hemoglobin (e.g. O₂Hb, deoxyhemoglobin, MetHb, and/or CO—Hb). In embodiments, multiple wavelength meters may be used to measure the total hemoglobin content including at least some of the dyshemoglobins such as CO-Hb and/or MetHb. In some embodiments, the meter uses wavelengths ranging from 600 to 1400 nm in order to account for the major strands of hemoglobin.

In embodiments, the meter uses spectrophotometry and conductivity (using the varying conductivity of blood at different RBC concentrations) based methods to measure hemoglobin concentration.)

In other embodiments, the hemoglobin meter may use differential light absorption before and after blood flow obstruction to determine the hemoglobin level non-invasively.

Exemplary devices include, but are not limited to the Pronto, Pronto-7, Rainbow and Radical monitors (Masimo Corporation, Irvine, Calif.) and the SpectOLight sensor or NBM-200MP monitor (Orsense, Nes Ziona, Israel). It will be appreciated that the meter or monitor may measure or detect further biological parameters including, but not limited to oxygen content, pulse rate, perfusion index, etc.

In a further embodiment, the hematocrit is measured directly. For example, the Crit-Scan® (Haemonetics Corp.) is a non-invasive device that measures the hematocrit through the skin using a photo-optical array.

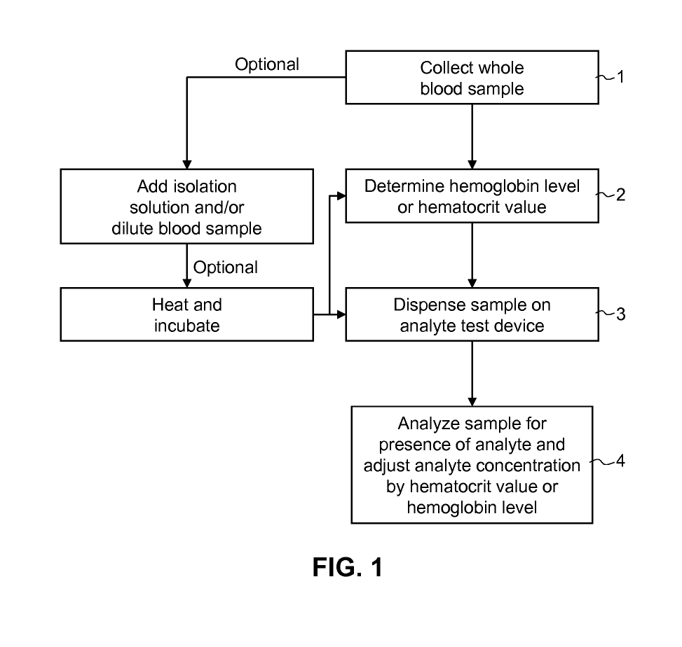

FIG. 1 illustrates the sequence of events for an assay method in one embodiment. In a first step, a whole blood sample is obtained from a subject 1. In an embodiment, the sample may be obtained by a fingerstick method, which generally involves using a lancet to obtain a venous blood sample. In an embodiment, about 20-50 μL or more of a blood sample is collected. In another embodiment, at least about 20 μL is collected. Typically up to about 500 μL may be obtained using fingerstick methods. The blood is collected with a pipette, capillary tube, or other suitable collection device.

Next, the hemoglobin level and/or hematocrit value of the blood sample or of the subject's blood is determined 2. In one embodiment, the hemoglobin level and/or hematocrit value is determined using the blood sample obtained from the subject in the preceding step. In another embodiment, the hemoglobin level and/or hematocrit value is determined using a non-invasive instrument. The hemoglobin level and/or hematocrit value can be determined prior to, concurrently with, or subsequent to obtaining the blood sample from the subject for analyte testing. That is, the hemoglobin level and/or hematocrit value may be measured, calculated or determined before or after the sample is obtained and/or at any point during the method. The hemoglobin level or hematocrit value is used to adjust for the whole blood effect. Where the hematocrit value is used for the adjustment, the hematocrit value may be measured directly from a whole blood sample, measured directly using a non-invasive meter that calculates hematocrit level of a subject's blood, calculated prior to entry into a device that measures the analyte concentration in the whole blood sample, or calculated from the hemoglobin level by the hemoglobin meter or by the device that measures the analyte concentration in the whole blood sample. In one embodiment, the hemoglobin level or hematocrit value is manually entered (e.g., via a user interface) into the instrument that reads the assay test device. In another embodiment, the device for measuring the hemoglobin level or hematocrit value communicates directly with the instrument that reads the assay test device. It will be appreciated that this communication may be through a physical connection, through a wireless connection, or via an online interface.

With continued reference to FIG. 1, the blood sample is dispensed onto an assay test device 3 and then the assay test device is analyzed for the presence of an analyte of interest 4. An exemplary instrument for optically reading an assay test device is described below. The instrument measures or detects the presence or level of analyte on the assay test strip and corrects for the whole blood effect using hemoglobin level and/or hematocrit value. Any one or more of the hemoglobin/hematocrit measurement device or the instrument that reads the assay test device may include or use an algorithm for determining a hematocrit value and/or adjusting a measured analyte level to account for the whole blood effect.

In a preferred embodiment of the method, the analyte level in the blood sample is quantitatively measured.

FIG. 1 also shows optional steps regarding processing of the blood sample. Subsequent to collecting the blood sample, an isolation reagent or solution may be added to the sample. In another embodiment, an isolation reagent or solution is included in a sample pad of a test device. An isolation reagent or solution may optionally be utilized when the analyte of interest is one that needs to be isolated or extracted from components within the blood sample. For example, some analytes are bound or associated with proteins or other components in the sample. It will be appreciated that the isolation reagent or solution may depend upon the particular assay being performed. For example, vitamin D must be released from vitamin D-binding protein in order to assay for vitamin D. Extraction and/or isolation reagents or solutions for analytes are known in the art. For example, organic solvents such as dichloromethane/methanol (Bouillon et al., Clin Chem, 22(3):364-368, 1976), acetonitrile, chloroform, hexane, ethanol, and ethyl acetate have been used as releasing reagents to separate vitamin D from its binding protein and/or other serum proteins. U.S. Pat. No. 8,003,400, incorporated herein by reference, describes several releasing or isolation reagents for vitamin D that may be used with the present assay. It will be appreciated that other extraction and/or isolation reagents or solutions are known in the art and may be used to isolate the analyte.

The sample may optionally be heated. The sample may be heated using any suitable method including, but not limited to a heat block or heated bath. In some embodiments, the sample is heated for about 1-10 minutes. In other embodiments, the sample is heated for about 2-10 minutes, about 2-3 minutes, about 2-4 minutes, about 2-5 minutes, about 2-6 minutes, about 3-10 minutes, about 3-4 minutes, about 3-5 minutes, or about 5-10 minutes. It will be appreciated the sample may be heated for longer or shorter period of times depending on the analyte and the presence of an isolation solution.

### III. System

In one aspect a system comprised of a device for measuring, detecting or determining hemoglobin content or level and/or hematocrit value and a device for measuring or detecting an analyte are provided. In some embodiments, the device for measuring or detecting an analyte comprises an apparatus capable of optically detecting a signal. In other embodiments, the device for measuring or detecting an analyte comprises (i) an assay test device and (ii) an apparatus or instrument capable of detecting a signal emanating from the assay test device. In the description below, the assay test device is exemplified by a lateral flow immunoassay test strip, and is sometimes referred to as a test strip. It will be appreciated that the assay test device is not intended to be limited to the lateral flow immunoassay test device used to exemplify the system, and a skilled artisan will appreciate that other assay test devices, such as microfluidic devices, immunoassays other than lateral flow based immunoassays, are contemplated. In one embodiment, the instrument that detects a signal emanating from the assay test device is capable of optically detecting a signal, such as a fluorescent signal or a reflected signal.

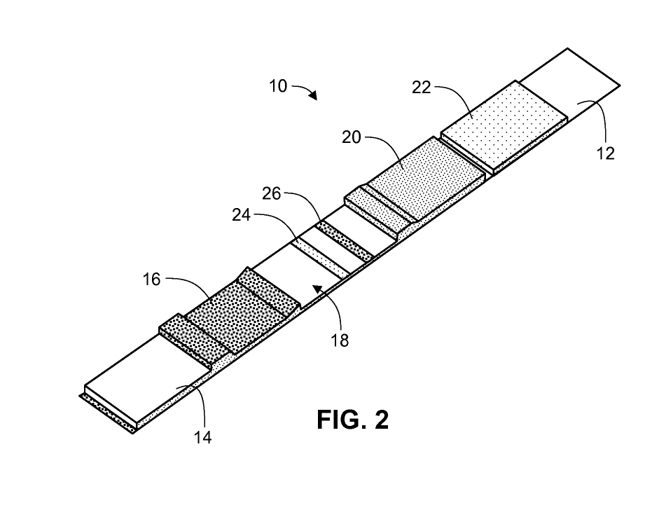

FIG. 2 is a perspective view of an assay test device, exemplified in this embodiment by a lateral flow immunoassay test strip 10. In the embodiment shown, test strip 10 is not situated within an external housing member, although it will be appreciated that the test strip can be contained within a housing, rigid or flexible, for improved handling by a user. Test strip 10 is comprised of a porous support member 12 that may extend the length of the test strip. The support member is generally made from any of a variety of materials through which the sample is capable of passing. For example, the material may be, but is not limited to, natural, synthetic, or naturally occurring materials that are synthetically modified, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; and the like.

Test strip 10 also comprises, in a downstream to upstream direction, at least some of a sample pad 14, a label pad 16, a detection zone 18, an absorbent pad 20, and an optional desiccant 22. Any or all of the sample pad, label pad, detection zone, absorbent pad, and desiccant may be positioned on the support member 12. A desiccant portion can be positioned on the support member of the test strip, and in one embodiment is disposed on the support member downstream of the absorbent pad, as described in U.S. Patent Application Publication No. 2008/0311002, incorporated by reference herein. The desiccant portion may be in contact with any of the sample pad, label pad, detection zone, or absorbent pad. In other embodiments, the desiccant portion is separate from at least one or all of the sample pad, label pad, detection zone, or absorbent pad. In another embodiment, a desiccant portion is a discrete component, physically separate from the test strip, inserted into a housing member that contains the test strip. Detection zone 18 is comprised of a test line 24 and, optionally, a reference line 26. The sample pad 14 is in fluid communication with the label pad 16 which is in fluid communication with the support member in the detection zone on which the test line and reference line are deposited. Some suitable materials that may be used to form the sample pad include, but are not limited to, nitrocellulose, cellulose, porous polyethylene pads, and glass fiber filter paper. If desired, the sample pad may also contain one or more assay pretreatment reagents, either diffusively or non-diffusively attached thereto. In an embodiment, the assay pretreatment reagent is an isolation solution or reagent used to separate an analyte from components within the sample such as binding proteins. The label pad 16 is formed from a material through which the sample is capable of passing. For example, in one embodiment, the label pad is formed from glass fibers. Although only one label pad is shown, it should be understood that multiple label pads may be present.

Deposited on the label pad are a first population of a labeled reagent with specific binding to the analyte of interest, and a second population of a labeled reagent with specific binding to an analyte that is not the analyte of interest; i.e., specific binding to an analyte other than the analyte of interest or, alternatively, specific binding to a specific analyte other than the analyte of interest. In one embodiment, the labeled reagent in the first and second populations comprises a collection of beads or particles (also referred to as microparticles) derivatized on their external surfaces with a respective specific binding member. For example, in one embodiment, the first population is a population of detectable particles capable of specific binding to an analyte of interest. The second population is a population of detectable particles capable of specific binding to an analyte other than the analyte of interest, and in one embodiment, capable of specific binding to a specific analyte other than the analyte of interest (also referred to as a non-test analyte).

The detectable substance to which the specific binding members are associated (ionically or covalently) may be a luminescent compound that produces an optically detectable signal. For example, suitable fluorescent molecules may include, but are not limited to, fluorescein, europium chelates, phycobiliprotein, rhodamine, and their derivatives and analogs. Other suitable fluorescent compounds are semiconductor nanocrystals commonly referred to as “quantum dots.” For example, such nanocrystals may contain a core of the formula CdX, wherein X is Se, Te, S, and so forth. Further, suitable phosphorescent compounds may include metal complexes of one or more metals, such as ruthenium, osmium, rhenium, iridium, rhodium, platinum, indium, palladium, molybdenum, technetium, copper, iron, chromium, tungsten, zinc, and so forth.

The detectable reagent, in one embodiment, is a compound that has a relatively long emission lifetime, and has a relatively large “Stokes shift.” The term “Stokes shift” is generally defined as the displacement of spectral lines or bands of luminescent radiation to a longer emission wavelength than the excitation lines or bands. A relatively large Stokes shift allows the excitation wavelength of a luminescent compound to remain far apart from its emission wavelengths and is desirable because a large difference between excitation and emission wavelengths makes it easier to eliminate the reflected excitation radiation from the emitted signal. Further, a large Stokes shift also minimizes interference from luminescent molecules in the sample and/or light scattering due to proteins or colloids, which are present with some body fluids (e.g., blood). In some embodiments, the luminescent compounds have a Stokes shift of greater than about 50 nanometers, in some embodiments greater than about 100 nanometers, and in some embodiments, from about 100 to about 350 nanometers. Exemplary fluorescent compounds having a large Stokes shift include lanthanide chelates of samarium (Sm (III)), dysprosium (Dy (III)), europium (Eu (III)), and terbium (Tb (III)). These chelates may exhibit strongly red-shifted, narrow-band, long-lived emission after excitation of the chelate at substantially shorter wavelengths. Typically, the chelate possesses a strong ultraviolet excitation band due to a chromophore located close to the lanthanide in the molecule. Subsequent to excitation by the chromophore, the excitation energy may be transferred from the excited chromophore to the lanthanide. This is followed by a fluorescence emission characteristic of the lanthanide. Europium chelates, for instance, have Stokes shifts of about 250 to about 350 nanometers, as compared to only about 28 nanometers for fluorescein. Also, the fluorescence of europium chelates is long-lived, with lifetimes of about 100 to about 1000 microseconds, as compared to about 1 to about 100 nanoseconds for other fluorescent labels. These chelates additionally have a narrow emission spectra, typically having bandwidths less than about 10 nanometers at about 50% emission. One suitable europium chelate is N-(p-isothiocyanatobenzyl)-diethylene triamine tetraacetic acid-Eu³.

The detectable substance may take the form of a particle or bead, and in particular a synthetic particle or bead. In one embodiment, latex particles that are labeled with a fluorescent or colored dye are utilized. In another embodiment, a particle with a fluorescent, phosphorescent or luminescent core coated with a polymer is utilized, the polymer surrounding the core typically formed from polystyrene, butadiene styrenes, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth.

The specific binding members attached to the particles may be antigens, haptens, aptamers, antibodies (primary or secondary), and complexes thereof, including those formed by recombinant DNA methods or peptide synthesis. An antibody may be a monoclonal or polyclonal antibody, a recombinant protein or a mixture(s) or fragment(s) thereof, as well as a mixture of an antibody and other specific binding members. Other common specific binding pairs include but are not limited to, biotin and avidin (or derivatives thereof), biotin and streptavidin, carbohydrates and lectins, complementary nucleotide sequences (including probe and capture nucleic acid sequences used in DNA hybridization assays to detect a target nucleic acid sequence), complementary peptide sequences including those formed by recombinant methods, effector and receptor molecules, hormone and hormone binding protein, enzyme cofactors and enzymes, enzyme inhibitors and enzymes, and so forth. Furthermore, specific binding pairs may include members that are analogs of the original specific binding member. For example, a derivative or fragment of the analyte (i.e., “analog”) may be used so long as it has at least one epitope in common with the analyte. Additionally, a binding protein that is specific for the analyte may be used as the specific binding member. For example, the vitamin D binding protein may be used as the specific binding member to detect vitamin D and/or metabolites, derivatives, and analogs thereof.

The specific binding members may generally be attached to the detectable particles using any of a variety of well-known techniques. For instance, covalent attachment of the specific binding members to the detectable particles may be accomplished using carboxylic, amino, aldehyde, bromoacetyl, iodoacetyl, thiol, epoxy and other reactive or linking functional groups, as well as residual free radicals and radical cations, through which a protein coupling reaction may be accomplished. A surface functional group may also be incorporated as a functionalized co-monomer because the surface of the detectable particles may contain a relatively high surface concentration of polar groups. In addition, although detectable particles are often functionalized after synthesis, such as with poly(thiophenol), the detectable particles may be capable of direct covalent linking with a protein without the need for further modification.

In one embodiment, the label pad of the immunoassay test strip comprises a first population of mobilizable detectable particles that bind specifically to an analyte of interest in a sample. The label pad may also include a second population of mobilizable detectable particles that bind specifically to an analyte other than the analyte of interest in a sample. In one example, the first population of particles comprises a monoclonal antibody for specific binding with a protein analyte of interest, such a human chorionic gonadotropin (hCG), and the second population of particles comprises an antibody with specific binding to immunoglobulin G. For example, each particle in the second population of particles is derivatized with an antibody with specific binding to the alpha chain of hCG. Each particle in the second population of particles is derivatized to comprise a goat anti-rabbit IgG antibody, for specific binding to IgG protein in the sample.

With continuing reference to FIG. 2, test line 24 comprises a binding member that is immobilized on the support membrane 12. The immobilized binding member in the test line serves to capture particles in the first population of particles that comprise a binding member specific for the analyte of interest, and in this way captures on the test line all or a portion of the detectable particles in the first population that have bound to the analyte of interest. The immobilized binding member preferably binds specifically to the analyte of interest at a location different from the binding site for the detectable particle and the analyte of interest. The immobilized binding member at the test line can be any of the binding members listed above, including an antibody, an antigen, a binding protein, and the like.

The detection zone in the test strip also comprises an optional reference line 26. Reference line 26 comprises a binding member that is immobilized on the support membrane 12. The immobilized binding member in the reference line serves to capture particles in the second population of particles that comprise a binding member specific for an analyte other than the analyte of interest, and in this way captures on the reference line all or a portion of the detectable particles in the second population. The immobilized binding member preferably binds specifically to a non-test analyte at a location different from the binding site for the detectable particle and non-test analyte. The immobilized binding member at the reference line can be any of the binding members listed above, including an antibody, an antigen, a binding protein, and the like.

The test strip may further include one or more reference lines used to (i) ascertain whether sample flow along the test strip occurred based on its RLU signal (emission), and (ii) for use by the analyzer (or more properly, an algorithm stored within the analyzer) to determine the relative locations of the other lines (control, if present, and analyte-specific test line(s)) on the test strip. The reference line is also used to ascertain a cut-off value, to render the immunoassay insensitive to incubation time, and in particular insensitive to incubation time over a period of 1-15 minutes, preferably 1-12 minutes, more preferably 1-10 or 2-10 minutes. Exemplary reference lines and uses thereof are described further in U.S. Patent Publication No. 2013/0230845, which is incorporated herein by reference.

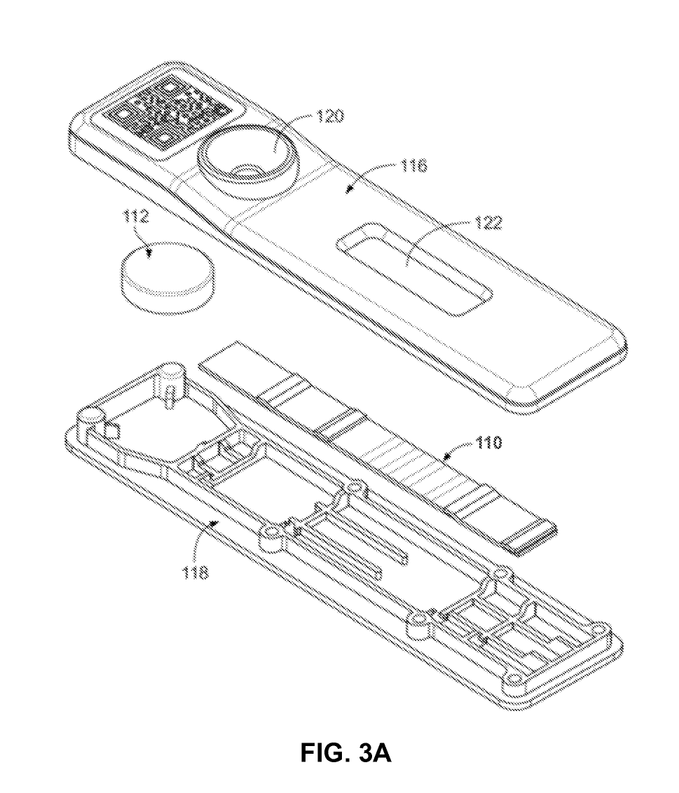

In one embodiment, the test strip is enclosed in a housing, sometimes referred to as a cassette, such as housing 114 in FIGS. 3A-3B. Together a test strip inserted into a housing forms an assay test device. It will be appreciated that the test strip may be fully or partially within the housing. For example, a portion of the test strip may extend from the housing to allow for dip-stick style sampling. Housing 114 in this embodiment is comprised of an upper member 116 and a lower member 118 that fit together to form a housing. Lower member 118 may include architectural features that define dimensioned regions for receiving the test strip 110 and the optional desiccant 112. Upper housing member 116 includes at least two openings, a first sample input port 120 and a viewing window 122. The sample input port is disposed directly above the sample pad on the test strip, so that a sample dispensed into the sample input port contact the sample pad for flow along the test strip. In the embodiment shown, the sample input port includes a bowl portion to receive a liquid sample into the port. The viewing window is positioned to reveal the lines in the test strip, so the optics system in the apparatus can interact with the lines, as will be described below. It will be appreciated that the housing may also be formed of a single piece.

In an embodiment, a bar code label 124 is affixed to the housing member for providing information to a reader apparatus. Use of a bar code label and reader is described in U.S. Publication Nos. 2013/0230844 and 2013-0230845, which are incorporated herein by reference.

It will be appreciated that the assay test devices illustrated in FIGS. 2-4 are exemplary of lateral flow test devices in general. The test strip can be configured uniquely for any given analyte, and the external housing is optional, and if present, need not be a cassette housing but can be a flexible laminate, such as that disclosed in U.S. Patent Application Publication No. 2009/02263854 and shown in Design patent No. D606664, which are both incorporated by reference herein.

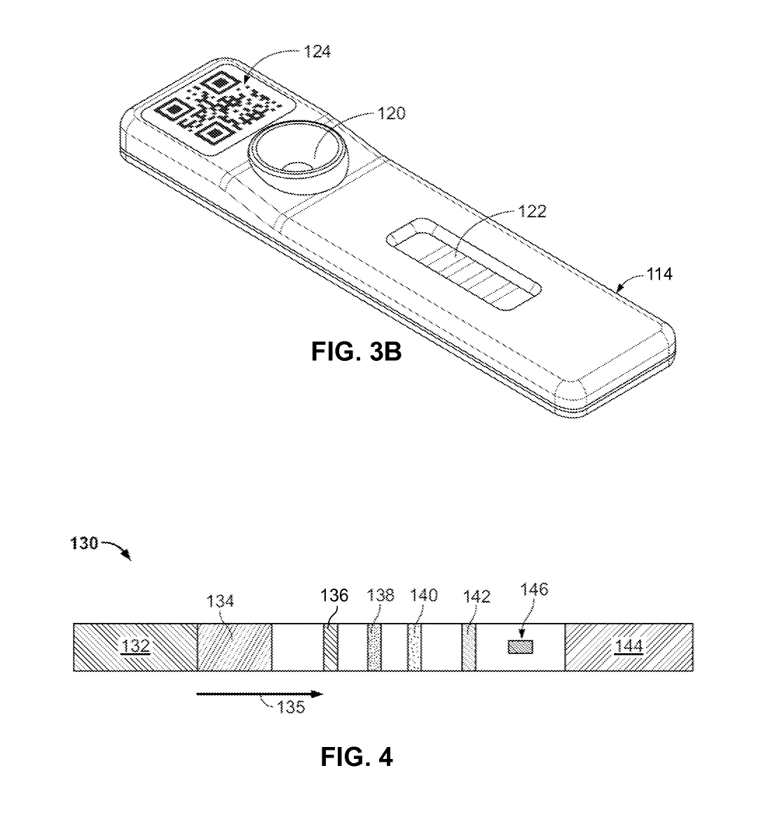

FIG. 4 is a top view of an exemplary test strip and the arrangement of its structural and immunochemical features for interaction with a detection apparatus. Test strip 130 includes a sample receiving zone 132 in fluid communication with a label zone 134. A fluid sample placed on or in the sample zone flows by capillary action from the sample zone in a downstream direction, indicated by arrow 135. Label zone 134 is in fluid communication with at least a test line and a control line or a reference line. In the embodiment shown in FIG. 4, the label zone is in fluid communication with a negative control line 136, an analyte test line 138, an optional second analyte test line 140, a reference line 142. The two or more lines are in fluid communication with an absorbent zone 144. That is, the label zone is downstream from the sample zone, and the series of control and test lines are downstream from the label zone, and the absorbent pad is downstream from the portion of the test strip on which the lines are positioned. A region between the downstream edge of the most downstream analyte-specific test line, which in the embodiment shown in FIG. 4 is test line 140, and the upstream edge of the absorbent pad is a procedural control zone (PCZ) 146. Reference line 142 is within the procedural control zone 146. The procedural control zone, and in particular the reference line therein, (i) ascertains whether sample flow along the test strip occurred based on its RLU signal (emission), and (ii) is used by the algorithm to determine the relative locations of the other lines (control, if present, and analyte-specific test line(s)) on the test strip. Materials for construction of each of the zones is well known in the art, and includes, for example, a glass fiber material for the sample zone, a nitrocellulose material on which the two or more lines are positioned.

The sample zone receives the sample suspected of containing an analyte of interest and controls its flow into the label zone. The label zone contains one or more labels for binding with the analyte. Labels are known in the art and are described, for example in U.S. Publication No. 2013/0230844, which is incorporated herein by reference. In an embodiment, the label or labels are conjugates that are comprised of particles containing a lanthanide element. In a preferred embodiment, the lanthanide is a chelated europium. The microparticles, in one embodiment, have a core of a lanthanide material with a polymeric coating, such as a europium core with polystyrene coating. A binding partner for the analyte(s) of interest in the sample is/are attached to or associated with the outer surface of the microparticles. In one embodiment, the binding partner for the analyte(s) of interest is an antibody, a monoclonal antibody or a polyclonal antibody. In another embodiment, the binding partner for the analyte is a binding protein. A skilled artisan will appreciate that other binding partners can be selected, and can include complexes such as a biotin and streptavidin complex. Upon entering the label zone, the liquid sample hydrates, suspends and mobilizes the dried microparticle-binding partner conjugates and carries the conjugates together with the sample downstream on the test strip to the control or reference and test lines disposed on the nitrocellulose strip. If an analyte of interest is present in the sample, it will bind to its respective conjugate as the specimen and microparticles flow from the label zone onto the surface of the nitrocellulose. In the embodiment shown in FIG. 4, this flowing mixture will then encounter negative control line 136. The negative control line is comprised of mouse immunoglobulin (IgG) and enables detection of non-specific binding of the conjugates to the immunoglobulin, thus approximating the level of non-specific binding that will occur at the downstream test line(s). The signal generated at this negative control line is used to help ensure that high non-specific binding at the analyte-specific test line does not lead to false positive results.

As the sample and microparticle-binding partner conjugates continue to flow downstream, if antigen is present in the sample, the fluorescent microparticle-binding partner conjugate which now includes bound with antigen/analyte of interest, will bind to the test line(s). In some embodiments, a single test line is present on the test strip. In other embodiments, at least two, or two or more test lines are present on the strip. By way of example, and as detailed in Example 1, a test strip intended for detection and/or discrimination of vitamin D will include a test line to detect vitamin D, its metabolites, or analogs. In embodiments, a second, third, or further test line may be used to detect additional metabolites or analogs.

The microparticle-binding partner conjugates that do not bind to the negative control line or to a test line continue to flow by capillary action downstream, and the remaining sample encounters the reference line. The reference line is comprised of a binding reagent such as goat anti-mouse immunoglobulin, and at least a portion of microparticle-binding partner conjugates that reach the reference line will bind non-specifically to the binding reagent. Fluorescent signal generated at this line provides information, for example, about the flow of the sample and also can serve as a location marker to direct the apparatus to the precise other locations on the nitrocellulose that are to be scanned by the optics system, as will be described below. The remaining sample then flows downstream of the reference line into the procedure control zone 146 that is also scanned by the optics system and is used, for example, to confirm that adequate flow of the sample has occurred. The sample with any remaining microparticle-binding partner conjugate then flows on into the absorbent pad.

The system further includes a device for measuring or detecting the analyte on the test device. The detector device may be any suitable reader or analyzer appropriate for use with the test devices described herein. In an embodiment, the detector is an apparatus capable of detecting a signal produced by a test device. Exemplary suitable detectors are described in U.S. Patent Publication Nos. 2013/0230844 and 2013/0230845, which are incorporated herein by reference.

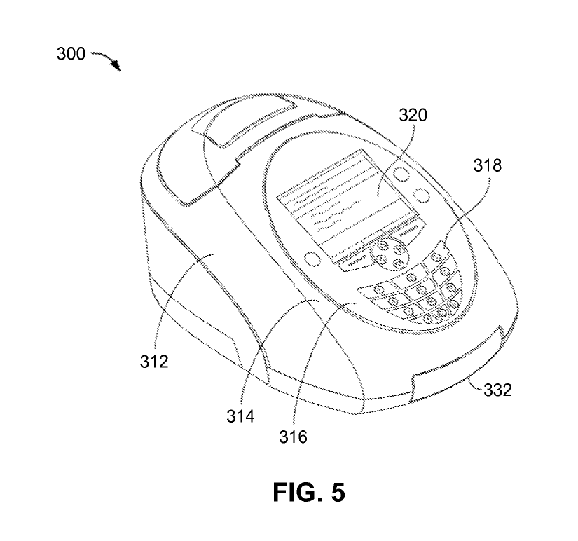

One exemplary apparatus is illustrated in FIG. 5. Apparatus 300 includes a housing 312 that encloses an optics system, electronics software, and other components of the apparatus. A front side 314 of the apparatus may include a user interface 316 that may include, for example, a key pad 318 and a display screen 320.

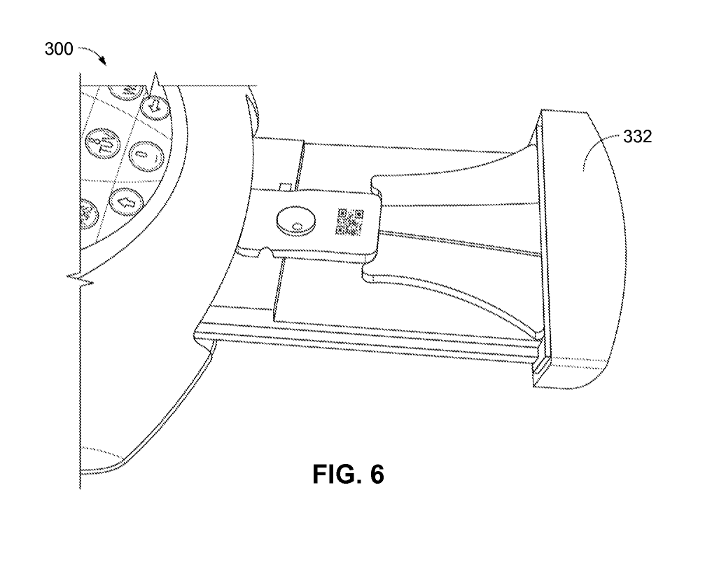

The apparatus preferably includes a drawer 332 movable between open and closed positions, as shown in FIG. 6 in an open position. The drawer is configured to receive a test device. Within the drawer, in one embodiment, is a distinct region, for example a depression, sized to receive the test device. During operation of the apparatus, the test device remains in a stationary position in the drawer, and therefore is positioned with precision in the apparatus for precise interaction with a movable optics system, described below. Accordingly, the drawer comprises in one embodiment a mechanism for positioning the test device for interaction with the optics system.

The apparatus may be equipped with ports for attachment to optional external devices. In one embodiment, the apparatus may include an attached external bar code scanner. The bar code scanner interfaces with the apparatus via a suitable data port provided on the apparatus. Externally attached devices ease transfer of data into and from the apparatus, and can eliminate user keyboard input, permitting accurate data input into the apparatus regarding a test to be analyzed or patient or sample information. In one embodiment, a barcode scanner external is attachable via PS-2 port on the apparatus and is capable of reading a linear or 1D bar code. In one embodiment, the bar code scanner is used to input data or information from the device for measuring, detecting or determining hemoglobin content and/or hematocrit value. The device for measuring, detecting or determining hemoglobin content and/or hematocrit value may directly prepare the bar code. Alternatively, the device for measuring, detecting or determining hemoglobin content and/or hematocrit value may communicate with a further device such as a bar code printer or a computer to prepare the bar code.

In one embodiment, the apparatus is wireless or wired connected to a device for delivering medical data to a third party, such as the Centers for Disease Control (CDC). In one embodiment, the apparatus communicates wirelessly with a further device such as a device for measuring or determining a hemoglobin content and/or hematocrit value. It will be appreciated that the data may be transferred to or from one or more cloud storage sites. In one specific non-limiting embodiment, data from the apparatus is wirelessly transmitted to an appropriate cloud. In another embodiment, data from a device for measuring or determining hemoglobin content and/or hematocrit value is transferred to a cloud where it is transferred to the detection device. In another embodiment, data from the device for measuring or determining hemoglobin content and/or hematocrit value is transferred directly to the detection device (e.g. through a wireless connection). In yet another embodiment, data from the device for measuring or determining hemoglobin content and/or hematocrit value generates a bar code containing the relevant information, which is then read by a bar code reader associated with the detection device.

The apparatus can include additional optional features, including for example acoustical output capability, to generate tones for audible feedback to a user, such as an error or test completion.

In an additional embodiment, the apparatus is wireless or wired connected to the device for measuring, detecting or determining hemoglobin content and/or hematocrit value. In this embodiment, the device for measuring, detecting or determining hemoglobin content and/or hematocrit value may directly communicate the hemoglobin level and/or hematocrit value to the apparatus for use in correcting or adjusting the measured or detected analyte level.

### IV. Analytes of Interest

The system comprised of an apparatus and an assay test device as described herein is intended for detection of any analyte of interest. Analytes associated with a disorder or a contamination are contemplated, including biological and environmental analytes. Analytes include, but are not limited to, vitamins, proteins, haptens, immunoglobulins, enzymes, hormones, polynucleotides, steroids, lipoproteins, drugs, bacterial antigens, viral antigens. In one embodiment, a test device is intended for detection or measurement of vitamin D, metabolites thereof, or derivatives or analogs thereof. With regard to bacterial and viral antigens, more generally referred to in the art as infections antigens, analytes of interest include Streptococcus, Influenza A, Influenza B, respiratory syncytial virus (RSV), hepatitis A, B and/or C, pneumococcal, human metapneumovirus, and other infectious agents well known to those in the art.

In other embodiments, a test device intended for detection of one or more antigens associated with Lyme disease is contemplated. In another embodiment, an assay test device designed for interaction with the apparatus is intended for use in the field of women's health. For example, test devices for detection of one or more of fetal-fibronectin, chlamydia, human chorionic gonadotropin (hCG), hyperglycosylated chorionic gonadotropin, human papillomavirus (HPV), and the like, are contemplated.

The assay test devices are intended for receiving a wide variety of samples, including biological samples from human bodily fluids, including but not limited to nasal secretions, nasopharyngeal secretions, saliva, mucous, urine, vaginal secretions, fecal samples, blood, etc. In one embodiment the test device is intended for receiving a whole blood sample.

The test devices, in one embodiment, are provided with a positive control swab or sample. In another embodiment, a negative control swab or sample is provided. For assays requiring an external positive and/or negative control, the apparatus is programmed to request a user to insert into the apparatus a test device to which a positive control sample or a negative control sample has been deposited. Kits provided with the test device can also include any reagents, tubes, pipettes, swabs for use in conjunction with the test device.

### V. Examples

The following examples are illustrative in nature and are in no way intended to be limiting.

### Example 1

### Detection and Discrimination of Vitamin D

A lateral flow test device comprised of a test strip and a housing is prepared. The test strip is fabricated with a sample pad comprised of a glass fiber matrix in fluid connection with a nitrocellulose strip, one or both supported on a support membrane.

Using standard NHS/carboxyl chemistry, vitamin D binding protein is covalently bound to the surface of europium chelate (.beta.-diketone)-incorporated polystyrene beads to form fluorescent microparticle-binding protein conjugates. The microparticle-antibody conjugates are deposited on a glass fiber matrix to form a label pad. The label pad is positioned adjacent the sample pad in a downstream direction. Two or more populations of microparticle-binding protein conjugates may be formed and deposited with the different conjugates directed to different metabolites or analogs of vitamin D may be prepared and deposited in the label pad.

An absorbent pad comprised of a highly absorptive material that acts as a wick to draw fluid from the nitrocellulose strip, thereby helping to ensure that adequate sample flow through the entire test strip is achieved, is positioned on the test strip downstream from the label pad and the nitrocellulose region.

The test strip is secured in a housing, for ease of handling. On an external upper surface of the housing is a bar code label containing information about the test strip, including for example, the intended analyte to be detected (vitamin D and/or metabolites or analogs), a device specific identification number, and an expiration date.

A blood sample is obtained from a patient using a fingerstick method. A finger is pricked with a lancet. At least about 20 μL of whole blood is obtained in a pipet, which is dispensed into a reaction tube or container. An isolation solution or reagent is added to the sample to release the analyte from vitamin D binding protein. The container is inverted one or more times to mix the sample and isolation reagent. The container is heated for 3-4 minutes in a heat block. The tube is inverted at least once and a portion of the test mixture is dispensed onto the sample pad via the sample input port in the housing.

Using the external barcode reader, the patient information is scanned into the apparatus or the information is entered using the keypad on the apparatus. The test device is into the drawer of the apparatus. The apparatus initiates its measurement sequence to scan the test device. The internal bar code scanner reads the information on the bar code label on the test device to determine the assay type, the device lot number, the test device serial number and the test device expiration date. The microprocessor loads the correct program into memory for the assay type to be run.

The microprocessor-controlled optics unit in the apparatus conducts its incremental, step by step scan of the length of the viewing window, which approximately corresponds to the length of the nitrocellulose region on the test strip. On the nitrocellulose strip the lines are sequentially read, beginning with the most downstream line, the reference line. The optics module moves relative to the stationary test device in an upstream direction to each of the analyte-specific test lines. At each incremental step, UV light from the UV LED is flashed on and then off. The UV light excites the europium fluorophore which in turn emits light at a wavelength.

After the apparatus completes its optical scan of the test window on the test device and collects the fluorescent data, it objectively interprets the assay result. A positive result for any analyte is determined by detection of a fluorescent signal at levels above a signal threshold set upon scanning the negative control line by a specific algorithm in the apparatus. The fluorescence signal obtained with this assay is invisible to the unaided eye. The test result can only be obtained with a fluorescent analyzer, which affords fully objective interpretation of the test result.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

We claim:

1. A method for measuring an analyte concentration in a whole blood sample, comprising:
measuring a hemoglobin content of a subject's blood or of a whole blood sample from a subject;
calculating a hematocrit value from the measured hemoglobin content;
measuring an analyte concentration in the whole blood sample with a measurement device; and
adjusting the measured analyte concentration based on the hematocrit value.

2. The method of claim 1, wherein measuring a hemoglobin content comprises non-invasively measuring a hemoglobin content.

3. The method of claim 2, wherein non-invasively measuring the hemoglobin content comprises non-invasively measuring using a hemoglobin meter.

4. The method of claim 3, wherein the hemoglobin meter comprises software comprising an algorithm to calculate a hematocrit value from the measured hemoglobin content.

5. The method of claim 3, wherein the hemoglobin meter is a pulse co-oximetry meter.

6. The method claim 1, wherein measuring the analyte concentration comprises a measurement device comprising an analyte test assay and an instrument to read a signal emanating from the analyte test assay to measure concentration of analyte, and the method further comprises providing the hemoglobin content to the instrument, whereby software on the instrument adjusts the measured analyte concentration based on the hemoglobin content.

7. The method of claim 6, wherein the hemoglobin content is wirelessly transmitted from the meter to the measurement device.

8. The method claim 4, wherein measuring the analyte concentration comprises a measurement device comprising an analyte test assay and an instrument to read a signal emanating from the analyte test assay to measure concentration of analyte, and the method further comprises providing hematocrit value to the instrument, whereby software on the instrument adjusts the measured analyte concentration based on the hematocrit value.

9. The method of claim 8, wherein hematocrit value is wirelessly transmitted from the meter to the measurement device.

10. The method of claim 1, wherein the analyte is vitamin D, a vitamin D metabolite, or a vitamin D derivative.

11. A method for measuring an analyte concentration in a whole blood sample, comprising:
measuring a hematocrit value of a subject's blood or of a whole blood sample;
measuring an analyte concentration in the whole blood sample with a measurement device; and
adjusting the measured analyte concentration based on the hematocrit value.

12. The method of claim 11, wherein measuring a hematocrit value comprises non-invasively measuring a hematocrit value.

13. The method of claim 11, wherein measuring a hematocrit value comprises measuring a hemoglobin level and calculating a hematocrit value.

14. The method of claim 12, wherein non-invasively measuring the hematocrit value comprises non-invasively measuring using a hematocrit meter.

15. The method of claim 11, wherein measuring the analyte concentration comprises a measurement device comprising an analyte test assay and an instrument to read a signal emanating from the analyte test assay to measure concentration of analyte, and the method further comprises providing the hematocrit value to the instrument, whereby software on the instrument adjusts the measured analyte concentration based on the hematocrit value.

16. The method of claim 11, wherein the hematocrit value is wirelessly transmitted from the meter to the measurement device.

17. The method of claim 11, wherein the analyte is vitamin D, a vitamin D metabolite, or a vitamin D derivative.

18. A system for measuring an analyte concentration in a whole blood sample, comprising:
a meter selected from (i) a hemoglobin meter for measuring a hemoglobin content of a subject's blood or of the whole blood sample; and (ii) a hematocrit meter for measuring a hematocrit value of a subject's blood or of the whole blood sample; and
a device for measuring the analyte concentration in the whole blood sample, the device comprising an instrument with (i) a user interface to input the measured hemoglobin content or hematocrit value and (ii) software to adjust a measured analyte concentration based on the measured hemoglobin content or hematocrit value;
wherein the device reports to a user an analyte concentration adjusted by the measured hemoglobin content or hematocrit value.

19. The system of claim 18, wherein the device includes an immunochromatographic test strip comprising a detection zone which contains an immobilized reagent capable of binding the analyte for detecting the analyte.

20. The system of claim 18, wherein the meter is a hemoglobin meter and the data corresponds to a measured hemoglobin content, and wherein the software comprises an algorithm to calculate a hematocrit value from the measured hemoglobin content.

21. The system of claim 18, wherein the meter is a hemoglobin meter that comprises an algorithm to calculate a hematocrit value from the measured hemoglobin content, wherein the hemoglobin meter reports or transmits the calculated hematocrit value to the device for measuring the analyte concentration in the whole blood sample.

22. A system for measuring an analyte concentration in a whole blood sample, comprising:
a meter selected from (i) a hemoglobin meter for measuring a hemoglobin content of a subject's blood or of the whole blood sample; and (ii) a hematocrit meter for measuring a hematocrit value of a subject's blood or of the whole blood sample, the meter comprising a wireless transmitter; and
a device for measuring the analyte concentration in the whole blood sample, the device comprising (i) a wireless receiver to receive data corresponding to a measured hemoglobin content or a measured hematocrit value from the meter and (ii) software to adjust a measured analyte concentration based on the data;
wherein the device reports an analyte concentration adjusted by the transmitted data.

23. The system of claim 22, wherein the device includes an immunochromatographic test strip comprising a detection zone which contains an immobilized reagent capable of binding the analyte for detecting the analyte.

24. The system of claim 22, wherein the meter is a hemoglobin meter and the data corresponds to a measured hemoglobin content, and wherein the software comprises an algorithm to calculate a hematocrit value from the measured hemoglobin content.

25. The system of claim 22, wherein the meter is a hemoglobin meter that comprises an algorithm to calculate a hematocrit value from the measured hemoglobin content, wherein the hemoglobin meter reports or transmits the calculated hematocrit value to the device for measuring the analyte concentration in the whole blood sample.
